(19) United States
(12) Patent Application Publication
(10) Pub. No.: US 2014/0311658 A1
(43) Pub. Date: Oct. 23, 2014

(54) METHOD OF MANUFACTURING DRESSINGS CONTAINING AT LEAST ONE ACTIVE INGREDIENT

(71) Applicant: LABORATOIRES URGO, Chenove (FR)

(72) Inventor: Denis FREROT, Saulon La Rue (FR)

(21) Appl. No.: 14/323,198

(22) Filed: Jul. 3, 2014

Related U.S. Application Data
(63) Continuation of application No. 13/002,728, filed on Feb. 9, 2011.

(30) Foreign Application Priority Data
Jul. 10, 2008 (FR) 0854685

(51) Int. Cl. A61F 13/02

(52) U.S. Cl. CPC A61F 13/0289
USPC 156/199

(57) ABSTRACT

A method of manufacturing dressings, preferably with thinner edges, is described. The method involves the steps of a) forming a first laminated element by securing together a backing film and a layer of a malleable adhesive material; b) shaping the laminated element to obtain an intermediate product that includes a plurality of zones each forming an incipient dressing; c) treating the resulting intermediate product in order to deposit one or more active ingredients on the free surface of the malleable adhesive material; d) forming a second laminated element by securing together the treated intermediate product and a protective film; and e) cutting the second laminated element into a plurality of dressings.

The present invention relates in general to a method for manufacturing wound dressings, in particular dressings having thinned edges, containing at least one active substance, said active substance being more particularly incorporated in the surface intended to be in contact with the wound and/or skin.

Dressings with thinned edges generally consist of a malleable adhesive material comprising, on one of its sides intended to be in contact with the wound and/or skin, a protective film forming a winglet which is detachable during use, and on the opposite side, a film forming a support.

Methods for manufacturing thinned edge dressings are known and described in particular in patents EP 0 573 708, EP 0 756 854, EP 0 818 187 and EP 0 999 025.

Thus, patent EP 0 573 708 describes a continuous method for manufacturing dressings comprising:
a first step in which a protective film and a malleable adhesive material are joined together and shaped in a molding station;
a second step in which a support film is complexed with the adhesive material on its side opposite the side bearing the protective film; and
a third step in which the combination thus formed is cut into individual dressings.

Patent EP 0 756 854 describes a continuous method for manufacturing dressings in which the various component layers (support film, malleable adhesive material and protective film) are joined and shaped simultaneously in a laminating-molding station supplied:
on the one hand, with a laminated element previously prepared by depositing the malleable adhesive material consisting of a mass containing hydrocolloids on a support film; and
on the other hand, with a protective film.

This method comprises a second step in which the combination previously shaped is cut into individual dressings.

Patent EP 0 818 187 describes a method for manufacturing dressings in which the various component layers are shaped and cut into individual dressings in the same step using a single workstation.

It should be observed that according to this document, the support film consists of a two-layer film whereof one of the layers is peelable and intended to protect the malleable adhesive material during its simultaneous shaping and cutting.

Patent EP 0 999 025 describes a method for manufacturing dressings that is similar to the method described in document EP 0 756 854, in which a two-layer support film comprising a delaminable layer is used in the first dressing shaping step and removed prior to the second individual dressing cutting step.

None of the methods described in these prior patents considers the incorporation of one or more active substances in the surface of the malleable adhesive material intended to be in contact with the wound and/or skin.

In fact, in each of the methods described in these patents, the protective film intended to form the detachable winglet of the dressing before use is joined to the malleable adhesive material prior to or during the molding step.

The implementation of these known methods for manufacturing a dressing containing at least one active substance would therefore require the prior incorporation of the active substance during the manufacture of the adhesive malleable material.

In fact, the adhesive malleable material is generally manufactured at elevated temperature, so that the incorporation of active substances in said material would be limited to substances that are unaffected by temperature.

Moreover, by incorporating the active substance in this step, it would be disseminated throughout the mass constituting the adhesive malleable material, and would therefore not be directly available at the wound.

Under these conditions, it is the object of the present invention to solve the technical problem of providing a novel method for manufacturing dressings, preferably with thinned edges, allowing the incorporation of one or more active substances in the surface intended to be in contact with the wound and/or skin, without affecting the adhesive properties thereof.

It is a further object of the present invention to solve the abovementioned technical problem in a manner that is simple and usable on the industrial scale.

Thus, the present application relates to a method for manufacturing wound dressings, preferably with thinned edges, characterized in that it comprises the steps consisting in:

a) forming a first laminated element by joining together a support film and a layer of an adhesive malleable material;

b) shaping said laminated element to obtain an intermediate product comprising a plurality of zones each forming a dressing preform;

c) treating the intermediate product thus obtained to deposit one or more active substances on the free surface of said adhesive malleable material;

d) forming a second laminated element by joining together said intermediate product thus treated and a protective film;

e) cutting said second laminated element thereby to form a plurality of dressings.

Said method has many advantages.

Firstly, the dressing obtained by the implementation of said method offers improved availability of the active substance, because the latter is directly present on the surface intended to be in contact with the wound and/or skin. Furthermore, the active substance can be deposited easily, localized on the surface of the dressing intended to cover the area to be treated.

Accordingly, the quantity of active substance(s) per dressing can be reduced, thereby limiting the manufacturing cost.

The support film used in the context of the present invention may consist of polymeric materials of varied types such as a polyamide, polyurethane, polyester, polyether, polyvinyl chloride, polyvinylidene chloride, polyvinyl alcohol, polyvinyl acetate, polystyrene, polyvinyl fluoride, a polyolefin such as, for example, a polyethylene or a polypropylene, a material based on polyether, polyester copolymer, polyether polyurethane copolymer, polyether polyamide copolymer.

Said support film is generally a thin film having a thickness between 5 and 150 μm, preferably between 15 and 70 μm.

Said support film may be adhesive or not on the side intended to be in contact with the adhesive malleable material.

Said support film may be monolayer or multilayer, such as for example a two-layer film whereof the second layer forms the surface opposite the one intended to be in contact with the adhesive malleable material. Said second layer may consist of a polymeric material of the same type as those mentioned above or may consist of silicone paper or other paper.

Advantageously, the support film consists of a two-layer film consisting of a non-adhesive polyurethane intended to be in contact with the adhesive malleable material complexed with a polyester or paper layer.

The support film may also consist of a nonwoven material or foam having a thickness of 10 to 500 μm.

In the context of the present description, “malleable material” means any material which, under the action of temperature (for example by heating) loses its elasticity.

Such materials may be of varied types and consist of materials commonly used for this purpose in dressings.

In general, said material may consist of an elastomeric matrix comprising hydrocolloids or not.

“Elastomeric matrix” means here compositions comprising:
one or more elastomers preferably selected from sequenced poly(styrene-olefin-styrene) copolymers;
one or more compounds from products called “tackifiers”, antioxidants, liquid plasticizers and cohesion improvers such as butyl rubbers or high molecular weight polyisobutylenes.

Such compositions are thus defined in “Advances in Pressure Sensitive Adhesive Technology” published by Donatas Satas in April 1995 in Chapter 7 “Wound dressings” pages 158 to 171 and are also perfectly known to a person skilled in the art.

Advantageously, in the context of the present invention, use is made of an adhesive malleable material consisting of an elastomeric matrix incorporating more or less large quantities of hydrocolloids, designated below by the expression “hydrocolloid masses”.

“Hydrocolloid” means here compounds used by a person skilled in the art for their ability to absorb hydrophilic liquids such as water, physiological serum or wound exudates.

As suitable hydrocolloids, mention can be made for example of pectin, alginates, natural plant gums such as karaya gum, cellulose derivatives such as carboxymethylcellulose and their salts of alkali metals such as sodium and calcium, and synthetic polymers based on salts of acrylic acid, known by the term “superabsorbents”, such as for example the products sold by BASF under the name Luquasorb® 1003 and by Ciba Specialty Chemicals by the name Salcare® SC91. Mixtures of these products can be used as hydrocolloids.

Alternatively, the adhesive malleable material used in the context of the present invention may consist of an oleogel or a silicone based material.

In the context of the present invention, “active substance” means any compound or composition having dermatological or cosmetic pharmacological properties that are useful for the treatment of the skin or for the treatment of wounds.

For example, mention can be made of anti-inflammatories, analgesics, antiseptics and antibacterials, agents promoting cicatrization or allowing cleaning of the wound, topic anesthetics, keratolytic agents. One or more agents belonging to the abovementioned classes can be used as active substances.

These active substances may be in various forms, and in particular in powder or liquid form (solution or dispersion).

These active substances may further be packaged in the form of microcapsules. These microcapsules preferably consist of a water soluble material capable of dissolving in the wound exudates to release the active substance(s) at the wound. The salting out kinetics of the active substance(s) can be controlled by the sound choice of the capsule material.

The particle size of the powders of microgranules of active substance(s) may vary to a large extent. In general, said particle size may be about 200 to 400 microns.

The particle size of microcapsules incorporating the active substance or substances may be between 1 μm and 1 mm, preferably between 150 and 600 μm, even more preferably being about 400 μm.

When a plurality of active substances are incorporated in the dressings of the invention, they can be used in a single form, such as for example a mixture of powders or a solution or dispersion, or in different forms, such as for example a powder and a liquid.

The quantity of active substance(s) incorporated depends on the type of active substance and can easily be determined by a person skilled in the art. In general, the quantity of active substance(s) incorporated is about 0.01 to 0.25 g of active substance(s) per square centimeter of active area of the dressing, that is, area intended to be in contact with the wound and/or skin.

The protective film used in the context of the present invention is intended to constitute a protective winglet of the adhesive malleable material (hydrocolloid mass) intended to be removed prior to the use of the dressing on the wound and/or skin.

This protective film may consist of varied materials such as the materials commonly used by a person skilled in the art to protect the adhesive surface of a dressing. It may in particular be a paper, a polyester, a polypropylene or a silicone cardboard.

The invention will be better understood and other features thereof will appear more clearly from reading the explanatory description that follows, with reference to the appended schematic drawings in which:

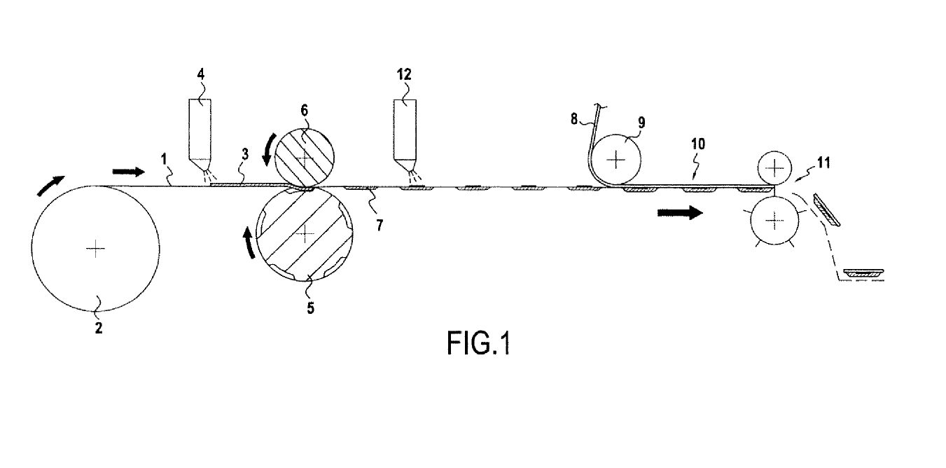

FIG. 1 schematically shows a first embodiment of the inventive method in which the dressings are manufactured continuously;

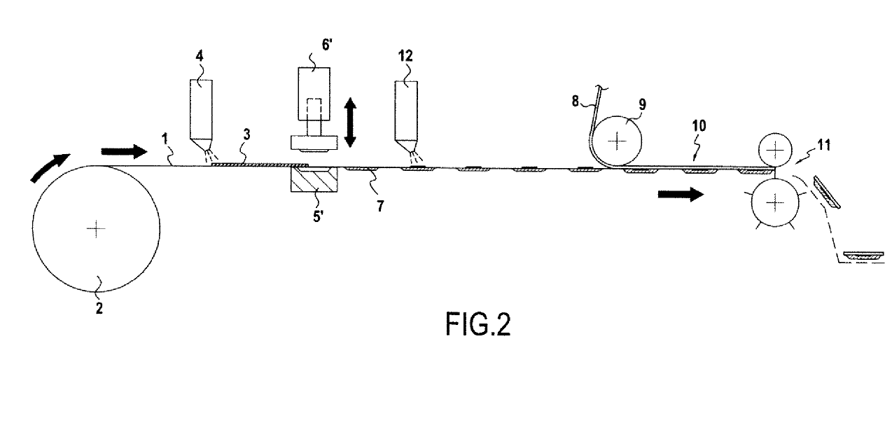

FIG. 2 schematically shows a second embodiment of the inventive method in which the dressings are manufactured in batches or sequentially.

In these figures, identical elements have the same reference numerals.

With reference to FIG. 1, a first embodiment of the inventive method is described in which the dressings are manufactured continuously.

A combination consisting of a support film 1 and an adhesive malleable material 3 is conveyed continuously to a “laminating-molding” station consisting of:
a first roll 5 comprising a set of cavities each having the general shape of the dressing to be obtained, preferably a dressing having thinned edges; and
a second roll 6 whereof the outer surface is substantially plane and smooth and consists of an anti-adhesive material.

In the example shown, the “support film-malleable material” combination supplied to the laminating-molding station is prepared by the deposition by coating, using the head 4 of a nozzle, of a malleable material 3 consisting of an elastomeric matrix optionally comprising hydrocolloids previously heated to a temperature of 100 to 160° C., on a support film supplied continuously by a feed spool 2.

When the combination thus formed passes through the laminating-molding station, the temperature of the adhesive malleable material is about 80 to 120° C.

According to an alternative embodiment, the adhesive malleable material can be extruded and conveyed directly to the rolls 5 and 6. In this case, the temperature of said material is about 70 to 90° C.

When they pass through the laminating-molding station, the adhesive malleable material 3 and the support film 1 are simultaneously joined together and shaped. It is obvious that this only represents a preferred embodiment, since the joining can be carried out during a prior step.

The roll 6 advantageously has a smooth surface consisting of an anti-adhesive material selected to prevent the adhesive malleable material 3 from adhering to said roll. For this purpose, the roll 6 can be covered with a silicone-based anti-adhesive substance.

Alternatively, the roll 6 may have convex shapes or cavities at its surface in order to form a slight depression on the surface of the dressing intended to accommodate the active substances and thereby facilitate their incorporation.

At the discharge end of the laminating-molding station, an intermediate product 7 is obtained comprising a plurality of zones having the general shape of the cavities present on the roll 5 and each forming a dressing preform, preferably with thinned edges.

The intermediate product 7 thus obtained is treated to deposit one or more active substances on the free surface of the portions comprising the adhesive malleable material 3.

This operation is advantageously carried out by powdering using a powder distributor 12 known to a person skilled in the art, when the active substance(s) is(are) in powder or microcapsule form.

According to an alternative embodiment, this operation can be carried out using a gun or a nozzle when the active substance(s) is(are) in liquid or semi-liquid form.

Advantageously, the active substance(s) is(are) deposited on the parts of the malleable material intended to constitute the central parts of the dressings. According to an alternative embodiment, the active substance(s) is(are) deposited in order to form patterns on the surface of the dressing such as, for example, a square, a rectangle, or one or more lines.

According to another alternative embodiment, the active substance(s) is(are) deposited in the form of pellets disposed in the zones intended to constitute the central parts of the dressings, or even distributed uniformly or not, on the surface of the zones intended to constitute the dressings. These pellets may have a diameter of about 6 to 8 millimeters for a dressing having a total surface area of about 1 to 3 cm², preferably 1.5 to 2.5 cm².

The intermediate product 7 thus treated is then joined to a protective film 8 continuously fed to a laminating roll 9. Said protective film is intended to constitute protective winglets detachable from the dressings before use.

A second laminated element 10 is thereby obtained.

Said second element is supplied continuously to a cutting station 11 known per se to form a plurality of individual dressings. FIG. 2 shows a second embodiment of the inventive method in which the dressings are manufactured in batches or sequentially.

Unlike the embodiment shown in FIG. 1, the laminating-molding station here comprises two plates movable toward one another, the first plate (5′) comprising a set of cavities each having the general shape of the dressing to be obtained, and a second plate (6′) whereof the surface consists of an anti-adhesive material. In the example shown, the first plate (5′) is fixed, only the second plate (6′) is movable longitudinally along an axis substantially perpendicular to the plane formed by the combination consisting of the film (1) and the malleable adhesive material (3). Obviously, other configurations are feasible, for example a configuration in which the two plates (5′, 6′) are movable longitudinally.

For the remainder, the said second embodiment is similar to the first embodiment described previously and serves to obtain the same dressings.

1-8. (canceled)

9. A method for manufacturing wound dressings, comprising:
a) forming a first laminated element by joining together a support film and a layer of an adhesive malleable material, the adhesive malleable material comprising an elastomeric matrix, a hydrophobic elastomer and at least one selected from the group consisting of a tackifying product, an antioxidant, a liquid plasticizer and a cohesion-improving agent, said elastomeric matrix comprising a hydrocolloid;
b) shaping said laminated element to obtain an intermediate product comprising a plurality of zones, each zone forming a dressing preform;
c) treating the intermediate product obtained in b) by depositing one or more active substances on the free surface of said adhesive malleable material so that the one or more active substances remains on the free surface of said adhesive malleable material, thereby forming a treated intermediate product in which the one or more active substances are on the free surface of said adhesive malleable material;
d) forming a second laminated element by joining together said treated intermediate product obtained in c) and a protective film; and
e) cutting said second laminated element, thereby forming a plurality of dressings,
wherein the steps a) and b) are carried out simultaneously in a laminating-molding station, and
wherein said active substance(s) is(are) deposited: (1) in a powder form using a powder distributor; or (2) in a paste or gel form, using a gun or a nozzle.

10. The manufacturing method as claimed in claim 9, wherein the laminating-molding station comprises a mold intended to be brought into contact with the support and an oddside mold comprising one side comprising an anti-adhesive material intended to be in contact with the adhesive malleable material.

11. The method as claimed in claim 10, wherein the laminating-molding station is supplied continuously and comprises a first roll having a plurality of cavities each having the general shape of the dressing to be prepared and a second roll whereof the outer surface is substantially plane and smooth and comprises an anti-adhesive material.

12. The manufacturing method as claimed in claim 10, wherein the laminating-molding station is supplied in batches and comprises two plates movable toward one another, the first plate comprising a set of cavities each having the general shape of the dressing to be obtained, and a second plate whereof the surface comprises an anti-adhesive material.

13. The method as claimed in claim 9 which comprises a step prior to the step a) of supplying the laminating-molding station with a combination formed of the support film in step a) bearing on one of its sides a layer of the adhesive malleable material in step a), said combination being obtained by coating, extrusion or by deposition.

14. A method for manufacturing wound dressings having thinned edges, comprising:
a) forming a first laminated element by joining together a support film and a layer of an adhesive malleable material, the adhesive malleable material comprising an elastomeric matrix, a hydrophobic elastomer and at least one selected from the group consisting of a tackifying product, an antioxidant, a liquid plasticizer and a cohesion-improving agent, said elastomeric matrix comprising a hydrocolloid;
b) shaping said first laminated element to obtain an intermediate product comprising a plurality of zones, each zone forming a dressing preform;
c) treating the intermediate product obtained in b) by depositing one or more active substances on the free surface of said adhesive malleable material so that the one or more active substances remains on the free surface of said adhesive malleable material, thereby forming a treated intermediate product in which the one or more active substances are on the free surface of said adhesive malleable material;
d) forming a second laminated element by joining together said treated intermediate product obtained in c) and a protective film; and
e) cutting said second laminated element, thereby forming a plurality of dressings, wherein said active substance is deposited: (1) in a powder form using a powder distributor, at the parts of the adhesive malleable material intended to constitute the central parts of the dressings; or (2) in a paste or gel form, using a gun or a nozzle, at the parts of the adhesive malleable material intended to constitute the central parts of the dressings.

15. The method as claimed in claim 9, wherein:
said elastomer is a poly(styrene-olefine-styrene)block copolymer; and
said cohesion-improving agent is at least one selected from the group consisting of a butylic rubber and a polyisobutylene having a high molecular weight.

16. The method as claimed in claim 9, wherein the cohesion-improving agent is a polyisobutylene.

17. The method as claimed in claim 9, wherein the elastomeric matrix is a poly(styrene-olefin-styrene) copolymer.

18. The method as claimed in claim 14, wherein the cohesion-improving agent is a polyisobutylene.

19. The method as claimed in claim 14, wherein the elastomeric matrix is a poly(styrene-olefin-styrene) copolymer.
